(19) United States
(12) Patent Application Publication
(10) Pub. No.: US 2019/0388694 A1
(43) Pub. Date: Dec. 26, 2019

(54) System and Method for Deep Brain Stimulation

(71) Applicant: Kara Stumpf, Fort Wayne, IN (US)

(72) Inventor: Kara Stumpf, Fort Wayne, IN (US)

(21) Appl. No.: 16/016,644

(22) Filed: Jun. 24, 2018

(51) Int. Cl. A61N 1/36; A61N 1/372; A61N 1/05

(52) U.S. Cl. CPC A61N 1/36142; A61N 1/0534; A61N 1/37211

(57) ABSTRACT

A deep brain stimulation system, said system comprising: a programmable neurostimulator, said neurotransmitter having a first output and a second output; a first electrical line, said first electrical line having a first color and functionally connected to said first outlet of said neurostimulator; a second electrical line, said second electrical line having a second color and functionally connected to said second outlet of said neurostimulator; a first deep brain stimulating lead, said first lead connected to said first electrical line; a second deep brain stimulating lead, said second lead connected to said second electrical line; said neurostimulator programmed to deliver a unique first signal through said first electrical line and a unique second signal through said second electrical line.

### BACKGROUND

### 1. Field of the Invention

The present invention relates to deep brain stimulation (“DBS”) and, more particularly, to a system and method for deep brain stimulation.

### 2. Description of the Related Art

DBS is a neurosurgical procedure introduced in 1987 involving the implantation of a medical device called a neurostimulator which sends electrical impulses, through implanted electrodes, to specific targets in the brain for the treatment of movement and neuropsychiatric disorders. DBS in select brain regions has provided therapeutic benefits for otherwise-treatment-resistant disorders such as Parkinson's disease, essential tremor, dystonia, chronic pain, major depression and obsessive-compulsive disorder.

The Food and Drug Administration (“FDA”) approved DBS as a treatment for essential tremor and Parkinson's disease in 1997, dystonia in 2003, and obsessive compulsive disorder (“OCD”) in 2009. DBS is also used in research studies to treat chronic pain, PTSD, and various affective disorders, including major depression. One example of a prior art DBS system is the Activa® system from Medtronic, Inc. The Activa®.

For many medical conditions, it is necessary to implant two or more leads in a patient's brain. Each position in the brain requires a unique signal (or set of pulses) to properly treat the patient. Instead of using multiple implanted neurotransmitters, it the current state of the art to for clinicians to employ a single programmable multichannel neurostimulator having more than one electrical outlet. Unfortunately, properly connecting an implanted lead to the correct electrical outlet on the neurotransmitter is often left to the memory of the clinicians performing a surgical procedure. There is no visual indication of the correct final assembly of the DBS system.

Thus, there is a continued need in the art for a system and method that can be utilized in a surgery during which DBS leads are being implanted to assist with safely, accurately, and reliably assuring the output signals of a neurotransmitter reach the correct lead.

### SUMMARY

DBS leads are placed in the brain according to the type of symptoms to be addressed. For non-Parkinsonian essential tremor, the lead is placed in the ventrointermediate nucleus of the thalamus; for dystonia and symptoms associated with Parkinson's disease (rigidity, bradykinesia/akinesia, and tremor), the lead may be placed in either the globus pallidus internus or the subthalamic nucleus; for OCD and depression to the nucleus accumbens; for incessant pain to the posterior thalamic region or periaqueductal gray; for Parkinson plus patients to two nuclei simultaneously, subthalamic nucleus and tegmental nucleus of pons, with the use of two pulse generators; and for epilepsy treatment to the anterior thalamic nucleus.

In one exemplary embodiment, the present invention may be described as a deep brain stimulation system, said system comprising: a programmable neurostimulator, said neurotransmitter having a first output and a second output; a first electrical line, said first electrical line having a first color and functionally connected to said first outlet of said neurostimulator; a second electrical line, said second electrical line having a second color and functionally connected to said second outlet of said neurostimulator; a first deep brain stimulating lead, said first lead connected to said first electrical line; a second deep brain stimulating lead, said second lead connected to said second electrical line; said neurostimulator programmed to deliver a unique first signal through said first electrical line and a unique second signal through said second electrical line.

In another exemplary embodiment, the present invention may be described as a method of performing deep brain stimulation in a medical context, said method comprising the steps of: programming a neurostimulator to send a first signal and a second signal, where said neurotransmitter has a first output and a second output; attaching a first electrical wire having a first color to said neurotransmitter at said first outlet; attaching said first electrical wire to a first deep brain stimulating lead; attaching a second electrical wire having a second color to said neurotransmitter at said first outlet; attaching said second electrical wire to a first deep brain stimulating lead; placing said leads into a brain at predetermined locations; and activating said neurotransmitter.

In another exemplary embodiment, the present invention may be described as a method of providing a deep brain stimulation system, said method comprising the steps of: providing a programmable neurostimulator, said neurotransmitter having a first output and a second output, said neurostimulator programmable to send a unique first signal through said first output and a unique second signal through said second output; providing a first electrical line, said first electrical line having a first color where said first electrical line can be functionally connected to said first outlet of said neurostimulator; providing a second electrical line, said second electrical line having a second color where said second electrical line can be functionally connected to said second outlet of said neurostimulator; providing a first deep brain stimulating lead where said first lead can be functionally connected to said first electrical line; providing a second deep brain stimulating lead where said second lead can be functionally connected to said second electrical line.

### BRIEF DESCRIPTION OF THE DRAWINGS

The present invention will be understood more fully from the detailed description given hereinafter and from the accompanying drawings of the preferred embodiment of the present invention, which, however, should not be taken to limit the invention, but are for explanation and understanding only.

In the drawings:

FIG. 1 shows an exemplary embodiment of a system according to the present invention in use on a patient.

FIG. 2 shows an exemplary multichannel, programmable neurotransmitter device in accordance with the present invention.

FIG. 3 shows a flow chart of an exemplary method of the present invention.

FIG. 4 shows a flow chart of another exemplary method of the present invention.

Corresponding reference characters indicate corresponding parts throughout the several views. The exemplary embodiments set forth herein are not to be construed as limiting the scope of the invention in any manner.

### DETAILED DESCRIPTION OF THE EMBODIMENTS

The present invention will be discussed hereinafter in detail in terms of various exemplary embodiments according to the present invention with reference to the accompanying drawings. In the following detailed description, numerous specific details are set forth in order to provide a thorough understanding of the present invention. It will be obvious, however, to those skilled in the art that the present invention may be practiced without these specific details. In other instances, well-known structures are not shown in detail in order to avoid unnecessary obscuring of the present invention.

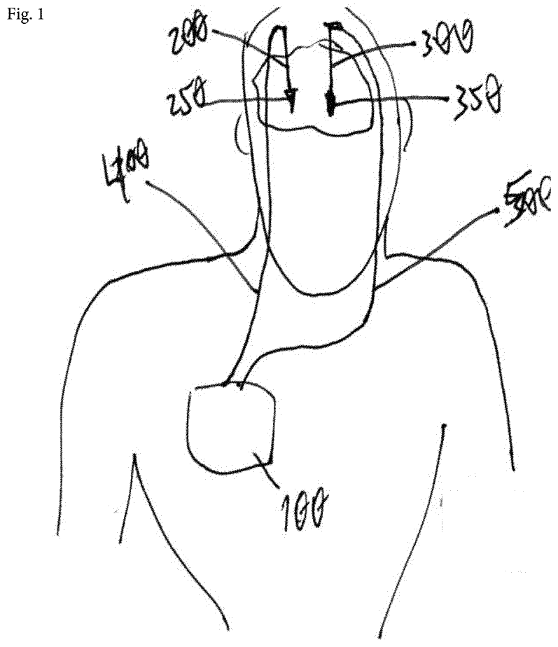

Thus, all of the implementations described below are exemplary implementations provided to enable persons skilled in the art to make or use the embodiments of the disclosure and are not intended to limit the scope of the disclosure, which is defined by the claims. As used herein, the word “exemplary” or “illustrative” means “serving as an example, instance, or illustration.” Any implementation described herein as “exemplary” or “illustrative” is not necessarily to be construed as preferred or advantageous over other implementations. Moreover, in the present description, the terms “upper”, “lower”, “left”, “rear”, “right”, “front”, “vertical”, “horizontal”, and derivatives thereof shall relate to the invention as oriented in FIG. 1.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Referring first to FIG. 1, the deep brain stimulation (“DBS”) system 1000 of the present invention generally comprises implantable pulse generating neurotransmitter 100, a first implantable lead 200, a second implantable lead 300, a first extension wire 400, and a second extension wire 500.

Referring again to FIG. 1, neurotransmitter 100 is placed subcutaneously below the clavicle or, in some cases, the abdomen. Neurotransmitter 100 can be calibrated by a neurologist, nurse, or trained technician to optimize symptom suppression and control side-effects.

Referring again to FIG. 1, neurotransmitter 100 is a battery-powered neurostimulator encased in a titanium housing, which sends electrical pulses to the brain to interfere with neural activity at the target site. Neurotransmitter 100 further comprises a programmable microcontroller and electrical outlets 110 adapted to carry pulse signals from neurotransmitter 100.

Referring again to FIG. 1, lead 200 is a coiled wire insulated in polyurethane with four platinum iridium electrodes. Lead 200 is placed in a nucleus of the brain. Lead 200 is connected to neurotransmitter 100 by extension wire 400, an insulated wire that runs below the skin, from the head, down the side of the neck, behind the ear to implanted neurotransmitter 100.

Referring still to FIG. 1, lead 300 is a coiled wire insulated in polyurethane with four platinum iridium electrodes. Lead 300 is placed in a nucleus of the brain. Lead 300 is connected to neurotransmitter 100 by extension wire 500, an insulated wire that runs below the skin, from the head, down the side of the neck, behind the ear to implanted neurotransmitter 100.

Each lead 200 and 300 may further comprise axially spaced apart electrodes 250 or 350. In fact, leads 200 and 300 can have multiple exposed electrodes at the distal end that are connected to conductors which run along the length of lead 200 and 300 and connect to neurotransmitter 100. Each lead 200 and 300 is connected by wires 400 and 500 from the brain to the chest cavity connecting the electrodes with neurotransmitter 100.

All components of the present invention are surgically implanted inside the body using known methods. For example, leads 200 and 300 may be implanted under local anesthesia or with the patient under general anesthesia such as for dystonia. A hole of about 14 mm in diameter is drilled in the skull and the probe electrode is inserted stereotactically. During the awake procedure with local anesthesia, feedback from the patient is used to determine optimal placement of the permanent electrode. During the sleeping procedure, intraoperative magnetic resonance imaging (“MRI”) guidance is used for direct visualization of brain tissue and device. The installation of neurotransmitter 100 0 and extensions 400 and 500 may occur under general anesthesia. Generally, the right side of the brain is stimulated to address symptoms on the left side of the body and vice versa.

Returning to FIG. 1, although FIG. 1 presents an illustration of specific locations for leads 200 and 300, it is to be understood that the principles involved are equally applicable to other targets, as discussed above.

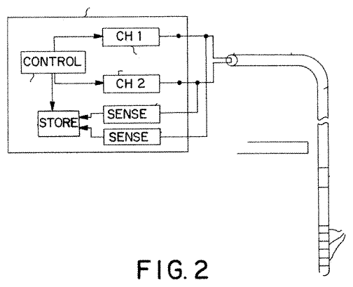

Referring now to FIG. 2, there is shown a diagram of neurotransmitter device 100 coupled to and used in connection with a lead 200 or 300. Transmitter 100 contains two pulse generation outputs (or “channels”), channel 1 indicated at 110 and channel 2 at 120. Each of these channels 110 and 120 is controlled by control block 130 that may appropriately utilize a microprocessor and/or other control and timing circuitry.

Referring again to FIG. 2, the proximal end of each lead 200 and 300 is connected to the outputs of transmitter 100. Channel 1 connects to lead 200, and channel 2 connects to lead 300 to provide electrical connections to distal electrodes 400 and 500 positioned at select locations within the patient's brain. Importantly, the present invention includes a visual indication selected from the group consisting of text, color, and exterior wire contour to demonstrate which extension wires 200 or 300 connect with each lead.

During surgery, if a surgeon implants leads in two locations, for example, he/she must be certain of which extension is for the left side and which extension is for the right side. Common procedure is for a surgeon to attach a silk suture to the left side This way, once the battery is attached, the surgeon will know which side is which. This is important information for the person programing the battery and so the patient can get the relief they need. To make it easier on the surgeon, the present invention comprises having a different color, texture, or labeling on each extension so that a surgeon can distinguish between sides.

A DBS procedure generally incorporates having an the following process. plan on arriving at the center an hour before your operative time. From the admitting area, you will be taken to a preoperative room where you will change clothes for surgery, have your blood pressure, pulse, and breathing checked, as well as be given the opportunity to take care of any bathroom needs. An intravenous line (IV) placed prior to surgery.

In the operating room, the neurosurgeon will then place a head frame onto the patient's skull. The box-like frame is necessary to precisely guide the neurosurgeon to the target. Because application of the head frame can be uncomfortable, local anesthesia is injected, which will numb the areas where the screws will attach to the skull to hold the head frame in place. Mild pain medication may also be offered. The head frame will remain in place for the entire surgery.

After the head frame is attached, a computed axial tomography (“CAT” scan”) or magnetic resonance imaging (“MRI”) will be done. This scan helps the neurosurgeon locate the exact site in the brain where the lead will be placed. The area where the skull will be opened may be shaved or cleaned with a special shampoo.

Alternatively, some surgical practices utilize a “frameless” DBS system. With this procedure, the neurosurgeon places several screws and plates in the patient's skull a day or more prior to surgery in order to hold the frameless system. In the operating room, the surgeon attaches recording equipment to the plates, and performs the surgery, essentially in an identical fashion to the frame based system. Individual neurosurgeons choose one method over the other based on experience, and individual preference.

After the brain scan, the patient's taken to the operating room. Besides the neurosurgeon, there will be nurses, and a movement disorders neurologist or neurophysiologist who will record the brain activity. The neurosurgeon often injects more anesthesia into the patient's scalp and then uses a drill to create a dime-sized hole in the skull, where a microelectrode will be inserted.

The microelectrode recording is a critical part of the surgery. This specialized procedure will guide the neurosurgeon in placing the lead in the exact area of the brain that will offer the best possible results. This part of the surgery can take a number of hours, depending upon the number of microelectrode passes required to pinpoint the target site, and also whether or not both sides of the brain are being operated on in one session. After the microelectrode recording locates the precise target, the neurosurgeon and neurologist will determine the best placement and put the permanent DBS lead in place.

After the lead is placed, the neurologist or neurosurgeon will connect it to an external generator, and administer brief electrical stimulation to observe if it improves the patient's symptoms. When it is confirmed that the lead is in the correct target region, the neurosurgeon fastens the permanent lead into place (with a capping device), and runs the connecting wire outside the brain and under the skin of the scalp. The hole in the skull is sealed with a plastic cap and stitches.

The implantable pulse generator (“IPG”) is often placed several weeks later and is performed as a separate surgery, but it may also be placed on the same day as the brain lead implantation. At that time, the connecting wire will be attached to the brain lead, and the IPG. This procedure takes about 40 minutes, and requires general anesthesia.

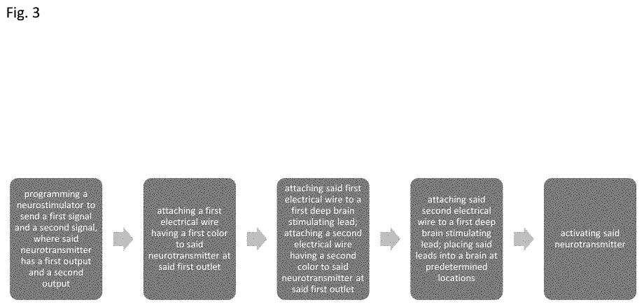

Referring now to FIG. 3, there is illustrated a flow diagram of the steps involved in an exemplary method the present invention. As illustrated in FIG. 3, these steps generally comprise programming a neurostimulator to send a first signal and a second signal, where said neurotransmitter has a first output and a second output; attaching a first electrical wire having a first color to said neurotransmitter at said first outlet; attaching said first electrical wire to a first deep brain stimulating lead; attaching a second electrical wire having a second color to said neurotransmitter at said first outlet; attaching said second electrical wire to a first deep brain stimulating lead; placing said leads into a brain at predetermined locations; and activating said neurotransmitter.

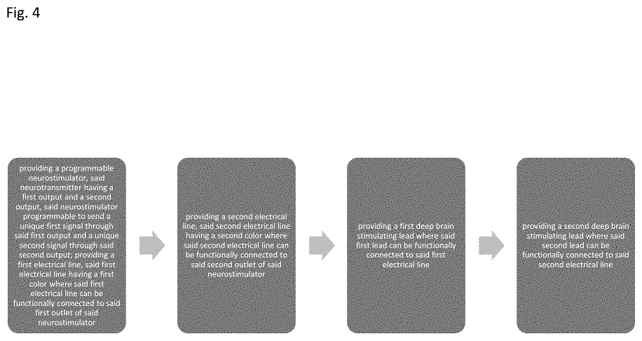

Referring now to FIG. 4, there is illustrated a flow diagram of the steps involved in another exemplary method the present invention. As illustrated in FIG. 4, these steps generally comprise the steps of: providing a programmable neurostimulator, said neurotransmitter having a first output and a second output, said neurostimulator programmable to send a unique first signal through said first output and a unique second signal through said second output; providing a first electrical line, said first electrical line having a first color where said first electrical line can be functionally connected to said first outlet of said neurostimulator; providing a second electrical line, said second electrical line having a second color where said second electrical line can be functionally connected to said second outlet of said neurostimulator; providing a first deep brain stimulating lead where said first lead can be functionally connected to said first electrical line; and providing a second deep brain stimulating lead where said second lead can be functionally connected to said second electrical line.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A deep brain stimulation system, said system comprising:
a programmable neurostimulator, said neurotransmitter having a first output and a second output;
a first electrical line, said first electrical line having a first color and functionally connected to said first outlet of said neurostimulator;
a second electrical line, said second electrical line having a second color and functionally connected to said second outlet of said neurostimulator;
a first deep brain stimulating lead, said first lead connected to said first electrical line; and
a second deep brain stimulating lead, said second lead connected to said second electrical line, where said neurostimulator programmed to deliver a unique first signal through said first electrical line and a unique second signal through said second electrical line.

2. A method of performing deep brain stimulation in a medical context, said method comprising the steps of:
programming a neurostimulator to send a first signal and a second signal, where said neurotransmitter has a first output and a second output;
attaching a first electrical wire having a first color to said neurotransmitter at said first outlet;
attaching said first electrical wire to a first deep brain stimulating lead;
attaching a second electrical wire having a second color to said neurotransmitter at said first outlet;
attaching said second electrical wire to a first deep brain stimulating lead;
placing said leads into a brain at predetermined locations; and
activating said neurotransmitter.

3. A method of providing a deep brain stimulation system, said method comprising the steps of:
providing a programmable neurostimulator, said neurotransmitter having a first output and a second output, said neurostimulator programmable to send a unique first signal through said first output and a unique second signal through said second output;
providing a first electrical line, said first electrical line having a first color where said first electrical line can be functionally connected to said first outlet of said neurostimulator;
providing a second electrical line, said second electrical line having a second color where said second electrical line can be functionally connected to said second outlet of said neurostimulator;
providing a first deep brain stimulating lead where said first lead can be functionally connected to said first electrical line; and
providing a second deep brain stimulating lead where said second lead can be functionally connected to said second electrical line.
